(19) United States
(12) Patent Application Publication
(10) Pub. No.: US 2013/0164316 A1
(43) Pub. Date: Jun. 27, 2013

(54) GENETIC SIGNATURES IN HIV-1 SUBTYPE C ENVELOPE GLYCOPROTEINS

(71) Applicants: Duke University, Durham, NC (US); Los Alamos National Security, LLC, Los Alamos, NM (US)

(72) Inventors: Barton F. HAYNES, Durham, NC (US); Feng Gao, Durham, NC (US); Bette T. Korber, Los Alamos, NM (US); David Montefiori, Durham, NC (US); Rosemary Musonda, Gaborne (BW)

(73) Assignees: Los Alamos National Security, LLC, Los Alamos, NM (US); Duke University, Durham, NC (US)

(21) Appl. No.: 13/671,490

(22) Filed: Nov. 7, 2012

Related U.S. Application Data
(63) Continuation of application No. PCT/US2011/000810, filed on May 9, 2011.
(60) Provisional application No. 61/332,262, filed on May 7, 2010.

(51) Int. Cl. C07K 14/16

(52) U.S. Cl. CPC C07K 14/162
USPC 424/188.1; 530/350; 536/23.72; 435/320.1; 530/387.9; 435/375

(57) ABSTRACT

The present invention relates, in general, to HIV-1 and, in particular, to immunogens that elicit broadly neutralizing antibodies against HIV-1 subtype C envelope glycoproteins, and compositions comprising same. The invention further relates to methods of inducing the production of such antibodies in a subject.

This application claims priority from U.S. Prov. Appln. No. 61/332,262, filed May 7, 2010, the entire content of which is incorporated herein by reference.

This invention was made with government support under Grant Nos. A1067854, A135351, and A164518 awarded by the National Institutes of Health. The government has certain rights in the invention.

### TECHNICAL FIELD

The present invention relates generally to HIV-1 and, in particular, to immunogens that elicit broadly neutralizing antibodies against HIV-1 subtype C envelope glycoproteins, to compositions comprising same, and to methods of inducing the production of such antibodies in a subject.

### BACKGROUND

The ability to elicit broadly cross-reactive neutralizing antibodies (Nabs) is an important goal for HIV-1 vaccines [1,2]. HIV-1 has nine genetically related lineages (subtypes A-K), and at a minimum at least one Glade should be effectively targeted in an HIV vaccine for that vaccine to be useful in a part of the world where a single subtype dominates the epidemic [3]. Nabs capable of targeting subtype C variants of the virus would be particularly useful, since subtype C accounts for approximately 50% of all HIV-1 infections world-wide [4], and the HIV epidemic in large regions of Southern Africa and in India is almost completely dominated by subtype C infections. Accordingly, there is a need to focus on subtype C antibody responses in natural infection. Efforts to generate cross-reactive Nabs have met with limited success, and novel approaches are urgently needed [1,5]. The potent cross-reactive neutralizing activity that is seen with a subset of human monoclonal Abs [6,7,8,9,10] and serum samples from HIV-1-infected individuals [11,12,13,14,15,16] is evidence that improvements in immunogen design may be possible.

Insights into how to make such improvements are being sought by studying autologous and heterologous Nabs in HIV-1-positive serum samples. Recent results indicate that epitopes in and around the CD4 binding site of gpl 20 comprise key targets for broadly neutralizing HIV-1-positive sera and that other key targets remain to be identified that play a substantial role [13,14,15]. Studies of autologous virus neutralization have shown that the response targets multiple regions of gp120, most notably epitopes in V1/V2 [17] and epitopes that require an interaction between C3 and V4 [17,18,19,20], A recent study identified 19 amino acid signatures in gp120 and gp41 that associated with the neutralization susceptibility of a multi-subtype panel of viruses [21]. Walker et al. have recently found two mAbs that bind to conformation determinants of HIV-1 Env and broadly neutralize about two-thirds of viruses tested [22].

Previous studies of the autologous and heterologous Nab response in HIV-1 infection have utilized one or limited number of representative env genes from each individual to characterize neutralization susceptibility [23,24,25,26]. Since env is highly variable in chronic HIV-1 infection (CHI) and because minor sequence changes can affect the biological function and antigenicity of the envelope glycoproteins [27,28,29,30,31,32], the study of a single env gene from each infected individual provides only a minimal representation of viral populations in vivo. Thus, depending on the design of the study, key information about neutralization epitopes may be missed under these conditions.

Another limitation of previous studies is that most have relied on a traditional bulk PCR methodology for Env cloning rather than single genome amplification (SGA) [23,24,25,26,33]. Viral sequences from the quasispecies population obtained by bulk PCR can result in artificial recombination and re-sampling as well as in nucleoside misincorporation by low fidelity Taq polymerase [34,35,36]. The SGA methodology makes it possible to obtain bona fide viral genomes from the infected individual [33,36,37,38]. Because viral sequences obtained by SGA more accurately reflect what is present in vivo [37], they can be used to better characterize viral gene functions.

The present study results, at least in part, from the use of SGA and a novel promoter PCR method to express functional Envs in a high throughput format [38] in order to explore the question of whether there are common neutralization signatures evident in Glade C viral sequences that associate with broad Nab responses. Multiple env genes from each of 37 HIV-1 infected individuals were obtained and characterized with respect to their infectivity and their susceptibility to neutralization by autologous and heterologous plasma samples. A signature sequence was found near the fourth variable region (V4) of gp120 that was associated with potent Nab responses in subtype C HIV-1-infected individuals.

### SUMMARY OF THE INVENTION

In general, the present invention relates to HIV-1. More specifically, the invention relates to immunogens that elicit broadly neutralizing antibodies against HIV-1 subtype C envelope glycoproteins, and to compositions comprising same. The invention further relates to methods of inducing the production of broadly neutralizing antibodies in a subject.

Objects and advantages of the present invention will be clear from the description that follows.

### BRIEF DESCRIPTION OF THE DRAWINGS

The patent application file contains at least one drawing executed in color. Copies of this patent application publication with color drawing(s) will be provided by the Office upon request and payment of the necessary fee.

FIG. 1. Phylogenetic tree analysis of newly characterized full-length env gene sequences. An unrooted phylogenetic tree was constructed with complete env gene sequences using the neighbor-joining method [54] and the Kimura two-parameter model [55]. Viruses with two lineages are indicated by underline; monophyletic env sequences are indicated by plain text. The branch lengths are drawn to scale (the scale bar represents 0.02 nucleotide substitutions per site).

FIG. 2. Phylogenetic tree analysis of clonal expansion env gene sequences. Midpoint-rooted phylogenetic trees were constructed with all env sequences from each individual who harbored clonal expansion viruses using the neighbor-joining method and the Kimura two-parameter model. Horizontal branch lengths are drawn to scale (the scale bar represents 0.005 nucleotide substitutions per site); vertical separation is for clarity only. Asterisks indicate bootstrap values in which the cluster to the right is supported in 85% or more replicates (out of 1,000). Patterns of identity or near identity are marked with black dots at the terminal leaves.

FIG. 3. Infectivity of Env pseudoviruses. The infectivity of 474 pseudoviruses from 37 individuals was determined in TZM-bl cells. The env genes were considered positive when the luciferase activity RLU values were 10-fold greater than those from SG3Δenv backbone control. The dotted line indicates this cutoff.

FIG. 4. Infectivity of pseudoviruses generated with recombinant env genes.

FIG. 5. Correlation between viral loads and infectivity of Env pseudovirsuses. Geometric means of the average luciferase activity of multiple Env pseudoviruses from each HIV-1 infected individual was plotted and analyzed by the Kendall's tau rank correlation coefficient method.

FIG. 6. Western blot analysis of HIV-1 protein expression in the transfected cells. The 293T cells transfected with pPCR products and pSG3Δenv were lysed 48 hrs after transfection. The viral proteins were separated on a 4-12% gradient reducing gel, transferred to nitrocellulose, and were reacted with an HIV-1 positive serum and a mouse mAb 13D5 to the Env protein. Viral proteins were visualized with secondary antibodies IRDye800 conjugated goat anti-human and Alexa-Fluor680 goat anti-mouse using a LiCor Odyssey Infrared Imaging system. The amounts of Env proteins (gp120 and gp41) are expressed as Env(gp160+gp120):P24 ratios. The infectivity of the each Env-pseudovirus is shown as relative light unit (RLU). Asterisks indicate the smaller Envs due to premature stop codons or non-inframe deletions.

FIG. 7. Maximum likelihood phylogenetic tree of the env sequences. All SGA env sequences from 15 HIV-1 infected individuals were included for analysis. Those were used for neutralization assays were indicated with colored marks at the tip of the tree branches. Envelopes were selected to be representative of the diversity in the sample. The sequences from samples with low diversity (monophyletic) are indicated in black; the sequences from samples with two distinctive phylogenetic clusters are colored so that those from one cluster are in red and those from the other are in blue; and sequences that represent recombinants between the two clusters are in purple.

FIG. 8. Neutralization analysis of Env pseudoviruses with autologous and heterologous plasmas. Sixty Env pseudoviruses from 15 patients were analyzed for their susceptibility to autologous and heterologous plasmas in a single round infection assay in TZM-bl cells. A purified HIVIg and an amphotropic murine leukemia Env pseudovirus (MLV-SVA) were included as a positive control and a non-specific neutralization control, respectively. Autologous neutralization with the contemporaneous plasma is indicated by a black box. The value in each cell is the plasma dilution at which viral replication was reduced 50% relative to the no plasma control. Titers <20 were assigned a value of 10. Red: >1,000; Orange: 500-1000; green: 100-499; yellow: 20-99; white: <20 (shown as 10) or nt (not tested).

FIG. 9. Hierarchical clustering of viruses and sera based on neutralization titers. Sera are clustered according to their ability to neutralize. Autologous responses are indicated in black boxes along the diagonal. The env SGA numbers are colored according to diversity: low diversity (black), two distinct groups (red and blue), and recombinant (purple). For ease of visualization, the heatmap is organized such that the rows (Env pseudovirues) are arranged according to individuals rather than hierarchical clustering, where hierarchical clustering patterns of the Env pseudoviruses are shown as a dendogram to the right of the figure. Clustering of high and low neutralization plasma was statistically supported, with a probability of 0.96 that the distinctive low-neutralization cluster was robust, using the approximately unbiased multistep-multiscale bootstrap re-sampling method developed by Shimodaira [43]. To illustrate this grouping, the columns are presented according to like-responses to the pseudoviruses based on the clustering hierarchy shown at the top of the figure. The sets of plasma with low (L) or high (H) NAb titers were grouped. Clustering patterns on the right represent significant unbiased multi-step multi-scale bootstrap re-sampling values 95 or greater. The columns were presented according to like-responses to the pseudoviruses based on the clustering hierarchy. The sets of plasma with low (L) or high (H) NAb titers were grouped, and the consensus envelope sequence from each individual classified and subsequently searched for signature sites that might predict the neutralizing potency of the individual's antibody response.

FIG. 10. The signature amino acid sequences associated with high levels of neutralization activity in plasma. H represents sequences in the group of plasma samples with high neutralizing activity against heterologous viruses, whereas L represents sequences that exhibited weak neutralizing activity. Numbers are used to show corresponding locations in the HXB2 reference strain. Dashes indicate the identical amino acids present in the reference sequence. Periods are used to designate gaps to maintain the alignment. Signature sites associated with potent neutralization are shown in red. Non-signature amino acids in key positions are shown in blue.

FIG. 11. Crystal structure of ligated gp120 with signature amino acids. The figure shows three signature sites, 393 (orange), 397 (magneta) and 413 (red) on a crystal structure of liganded gp120 (PDB:2B4C) [56]. The V4 loop and alpha-2 helix are marked in different colors for clarification. Positions in C3 that exhibit significant contact with these signature sites on V4 are also marked with the same color as the signature sites.

FIG. 12. Comparison of autologous neutralizing activity between low and high heterologous neutralizing plasma samples. Nab titers from low heterologous neutralization plasmas (6) or high heterologous neutralization plasmas (9) were compared. Values at Y-axis are the reciprocal plasma dilution at which luciferase activity (RLU) was reduced 50% relative to virus control wells.

### DETAILED DESCRIPTION OF THE INVENTION

An increase in knowledge of the molecular and antigenic structure of the HIV-1 envelope glycoproteins (Env) has yielded important new insights for vaccine design but translating this information to an immunogen that elicits broadly neutralizing antibodies has been difficult. The present invention is based, at least in part, from the use of SGA and a novel promoter PCR method to express functional Envs in a high throughput format [38] in order to explore the question of whether there are common neutralization signatures evident in Glade C viral sequences that associate with broad Nab responses. As described in the Example that follows, the neutralization properties of serum antibodies for autologous and heterologous multiple authentic and functional HIV-1 env genes in HIV-1 Glade C quasispecies from 34 chronically infected individuals were characterized. A total of 474 full-length HIV-1 env genes (5-23 per subject) were obtained by single genome amplification. One-third of these individuals harbored populations of clonally expanded viruses with identical or similar genetic sequences that accounted for 9%-38% of the total virus population. Functional analysis of these env genes as Env-pseudotyped viruses showed a wide range of virus infectivity. This variation in infectivity correlated with plasma viral load but not with protein expression levels in transfected cells, suggesting a more functional env gene may lead to the higher viral load in HIV-1 infected individuals.

The Example that follows also describes that neutralization susceptibility to antibodies in autologous and heterologous plasma samples was determined for multiple Envs (3-6) from each of 15 individuals. While reduced neutralization was observed for autologous viruses, heterologous neutralization was detected and could be divided into two distinct groups: plasma with broadly cross-reactive neutralization (N=9) and plasma that was poor at cross-reactive neutralization (N=6). Plasmas with stronger neutralizing activity against heterologous viruses more potently neutralized contemporaneous autologous viruses. Analysis of Env sequences in plasma from both groups revealed a three amino acid signature in the V4 region, proximal to the co-receptor binding site, that was associated with greater neutralization potency and breadth.

The present invention thus relates to HIV-1 subtype C Envs that retain the signature associated with potent antibody responses (e.g., signature sites 393, 397, and 413) and methods of using same as vaccine immunogens. The invention further relates to such Envs for use as diagnostic targets in diagnostic tests. The invention further relates to the use of wildtype (WT) virus sequences in the preparation of a polyvalent HIV-1 vaccine (U.S. Provisional Application No. 61/282,526, filed Feb. 25, 2010). Sequences that can be included in such a polyvalent vaccine for B cell response include env and for T helper and cytotoxic T cell response include gag, pol, nef and tat sequences (U.S. application Ser. No. 11/990, 222, filed Aug. 23, 2006).

The vaccine antigens (immunogens) of the invention (e.g. Envs sequences that retain the signature associated with potent antibody responses) can be chemically synthesized and purified using methods well known in the art. The immunogens can also be synthesized by well-known recombinant DNA techniques. Nucleic acids encoding the immunogens of the invention can be used as components of, for example, a DNA vaccine wherein the encoding sequence is administered as naked DNA or, for example, a minigene encoding the immunogen can be present in a viral vector. The encoding sequence can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric yin's vector, Salmonella species bacterial vector, Shigella species bacterial vector, Venezuelean Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the invention. Expression of the immunogen of the invention can be induced in a patient's own cells, by introduction into those cells of nucleic acids that encode the immunogen, preferably, using codons and promoters that optimize expression in human cells. Examples of methods of making and using DNA vaccines are disclosed in, for example, U.S. Pat. Nos. 5,580,859, 5,589,466, and 5,703,055.

The invention includes compositions comprising an immunologically effective amount of the immunogen of the invention (e.g., gp160 or gp140) or fragment thereof (e.g., gp41, gp120, either alone or associated with lipids, or fragments of gp120), or nucleic acid sequence encoding same, in a pharmaceutically acceptable delivery system. The compositions can be used for prevention and/or treatment of immunodeficiency virus infection (e.g., in a human). The compositions of the invention can be formulated using adjuvants (e.g., alum, AS021 (from GSK), oligo CpGs, MF59 or Emulsigen), emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for intranasal administration). The present compositions can be administered by any convenient route including subcutaneous, intranasal, intrarectal, intravaginal, oral, intramuscular, or other parenteral or enteral route, or combinations thereof. The immunogens can be administered in an amount sufficient to induce an immune response, e.g., as a single dose or multiple doses. Optimum immunization schedules can be readily determined by the ordinarily skilled artisan and can vary with the patient, the composition and the effect sought.

Examples of compositions and administration regimens of the invention include consensus or mosaic gag genes and consensus or mosaic nef genes and consensus or mosaic poi genes and consensus Env with an Env that retains the above-described signature or mosaic Env with an Env that retains the above-described signature, expressed as, for example, a DNA prime recombinant Vesicular stomatitis virus boost and a recombinant Env protein boost for antibody, a poxvirus prime such as NYVAC and a protein Env oligomer boost, or fragment thereof, or DNA prime recombinant adenovirus boost and Env protein boost, or, for just antibody induction, only the recombinant envelope gp120 or gp140 as a protein in an adjuvant. (See U.S. application Ser. No. 10/572,638, PCT/US2006/032907, U.S. application Ser. Nos. 11/990,222 and 12/192,015.)

The invention contemplates the direct use of both the immunogen of the invention and/or nucleic acid encoding same and/or the immunogen expressed as a minigene in the vectors indicated above. For example, a minigene encoding the immunogen can be used as a prime and/or boost,

It will be appreciated from a reading of this disclosure that the whole Envelope gene can be used or portions thereof (i.e., as minigenes). In the case of expressed proteins, protein subunits can be used.

As pointed out above, the invention also relates to diagnostic targets and diagnostic tests. For example, a signature-retaining Env of the invention can be expressed by transient or stable transfection of mammalian cells (or they can be expressed, for example, as recombinant Vaccinia virus proteins). The protein can be used in ELISA, Luminex bead test, or other diagnostic tests to detect antibodies to the transmitted/founder virus in a biological sample from a patient at the earliest stage of HIV infection.

The present invention also relates to antibodies specific for signature-retaining Envs of the invention, and fragments of such antibodies, and to methods of using same to inhibit infection of cells of a subject by HIV-1. The method comprises administering to the subject (e.g., a human subject) the HIV-1 specific antibody, or fragment thereof, in an amount and under conditions such that the antibody, or fragment thereof, inhibits infection.

In accordance with the invention, the antibodies can be administered prior to contact of the subject or the subject's immune system/cells with HIV-1 or after infection of vulnerable cells. Administration prior to contact or shortly thereafter can maximize inhibition of infection of vulnerable cells of the subject (e.g., T-cells).

As indicated above, either the intact antibody or fragment (e.g., antigen binding fragment) thereof can be used in the method of the present invention. Exemplary functional fragments (regions) include scFv, Fv, Fab′, Fab and F(ab′)₂ fragments. Single chain antibodies can also be used. Techniques for preparing suitable fragments and single chain antibodies are well known in the art. (See, for example, U.S. Pat. Nos. 5,855,866; 5,877,289; 5,965,132; 6,093,399; 6,261,535; 6,004,555; 7,417,125 and 7,078,491 and WO 98/45331.)

The antibodies, and fragments thereof, described above can be formulated as a composition (e.g., a pharmaceutical composition). Suitable compositions can comprise the antibody (or antibody fragment) dissolved or dispersed in a pharmaceutically acceptable carrier (e.g., an aqueous medium). The compositions can be sterile and can in an injectable form. The antibodies (and fragments thereof) can also be formulated as a composition appropriate for topical administration to the skin or mucosa. Such compositions can take the form of liquids, ointments, creams, gels, pastes or aerosols. Standard formulation techniques can be used in preparing suitable compositions. The antibodies can be formulated so as to be administered as a post-coital douche or with a condom.

The antibodies and antibody fragments of the invention show their utility for prophylaxis in, for example, the following settings:

i) in the setting of anticipated known exposure to HIV-1 infection, the antibodies described herein (or binding fragments thereof) can be administered prophylactically (e.g., IV or topically) as a microbiocide,

ii) in the setting of known or suspected exposure, such as occurs in the setting of rape victims, or commercial sex workers, or in any sexual transmission with out condom protection, the antibodies described herein (or fragments thereof) can be administered as post-exposure prophylaxis, e.g., IV or topically, and

iii) in the setting of Acute HIV infection (AHI), antibodies described herein (or binding fragments thereof) can be administered as a treatment for AHI to control the initial viral load and preserve the CD4+T cell pool and prevent CD4+T cell destruction.

Suitable dose ranges can depend, for example, on the antibody and on the nature of the formulation and route of administration. Optimum doses can be determined by one skilled in the art without undue experimentation. Doses of antibodies in the range of 10 ng to 20 μg/ml can be suitable.

The present invention also includes nucleic acid sequences encoding the antibodies, or fragments thereof, described herein. The nucleic acid sequences can be present in an expression vector operably linked to a promoter. The invention further relates to isolated cells comprising such a vector and to a method of making the antibodies, or fragments thereof, comprising culturing such cells under conditions such that the nucleic acid sequence is expressed and the antibody, or fragment, is produced.

Certain aspects of the invention can be described in greater detail in the non-limiting Example that follows. (U.S. Application Ser. Nos. 61/322,725, 61/322,821, and 61/322,663 are incorporated herein by reference.)

### EXAMPLE

Elicitation of broad and potent neutralizing antibodies to HIV-1 has been challenging. However, recent studies indicate that neutralizing antibodies are required for an AIDS vaccine to effectively control HIV-1 infection. To understand the ability of HIV-1 to induce neutralizing antibodies during natural infection, comprehensive autologous and heterologous neutralization assays were performed using multiple Env-pseudoviruses from each subtype C infected individual, and identified a three amino acid signature in the V4 region, proximal to the co-receptor binding site, that was associated with greater neutralization potency and breadth. Identification of a signature for eliciting broadly reactive neutralizing antibody responses has important implications for the development of vaccine candidates capable of inducing neutralizing antibodies to HIV-1. These results also showed the presence of autologous neutralization in the contemporaneous plasmas with stronger neutralizing activity against heterologous viruses, clonal expansion of viruses in the majority of the chronically infected individuals, and a positive correlation between the infectivity of Env-pseudoviruses and viral loads in the infected individuals.

### Experimental Details

Amplification of HIV-1 env Genes.

Plasma samples were collected from 37 HIV-1 positive individuals enrolled in a study of contemporary HIV-1 strains in Ndola, Zambia. The study was approved by the ethics committee of the Tropical Disease Research Centre, the Duke University Institutional Review Board, and the National Institutes of Health. Viral RNA was extracted from the plasma and reverse transcribed into cDNA using Superscript III (Invitrogen; Carlsbad, Calif.). Multiple rev/env genes from each individual were obtained by using single genome amplification (SGA), followed by the addition of a CMV promoter to the 5′ end of the SGA products using pPCR technology as previously described [38].

Single Round Infection Assay.

pPCR products were cotransfected with an env-deficient HIV-1 backbone plasmid (pSG3Δenv) into 293T cells in a 24 well plate using FuGENE6 transfection reagent (Roche Diagnostics; Indianapolis, Ind.). Briefly, pPCR DNA (150 ng) and pSG3Δenv DNA (150 ng) were mixed with 1.2 μl of FuGENE6 (FuGENE:DNA ratio at 3 μl:1 μg) in a total volume of 20 it with serum free DMEM, incubated for 30 minutes and added to 293T cells (70% confluence) seeded one day earlier at 5×10⁴ per well. Forty-eight hours after transfection, supernatants were harvested. Equal volumes of pseudovirions were added to TZM-bl cells with DEAE (5 μg/ml) in a 96 well plate (200 μl). Cultures were incubated for 48 hrs at 37° C. with 5% CO₂. Supernatants (100 μl) from infected TZM-bl cells were removed and 100 μl of Bright-Glo Luciferase Assay substrate with buffer (Promega; Madison, Wis.) was added to the cells. Following a 2-minute incubation, 100 μl of cell lysates were added to a solid black 96 well plate. Luminescence was measured with a Wallac 1420 Multilabel Counter (PerkinElmer: Waltham, Mass.). The TCID₅₀ was determined as described previously [39].

Neutralization Assay.

HIV-1 neutralization was measured as a reduction in luciferase activity after a single round infection of TZM-bl cells as previously described [39,40]. Equal amounts of pseudovirions (200 TCID₅₀) were used in each reaction. Neutralization titers against pPCR pseudovirions were determined for 16 plasma samples (15 autologous and 1 heterologous to the tested Env pseudoviruses) and one HIV-1 positive serum control. An amphotropic murine leukemia Env pseudovirus was also included as non-specific neutralization control.

Western Blot.

Forty-eight hours after cotransfection with pPCR products and pSG3Δenv, 293T cells were lysed with 250 μl of lysis buffer (50 mM Tris-HCl, 150 mM NaCl, 20 mM EDTA, 1% Triton-X100, 0.1% SDS, pH 7.4). Cell lysates were mixed with 6× denaturing sample buffer (300 mM Tris-HCl pH 6.8, 12 mM EDTA, 12% SDS, 864 mM 2-mercaptoethanol, 60% glycerol, 0.05% bromophenol blue). Samples were boiled for 10 minutes and then loaded on a NuPAGE Novex 4-12% Bis-Tris ge (Invitrogen; Carlesbad, Calif.). After the samples were transferred to a nitrocellulose membrane, the membrane was blocked in PBS containing 1% casein and 0.01% NaN₃ for 1 hr. The blot was reacted with an HIV-1 positive serum (1:500) and a mouse mAb 13D5 (1 μg/ml) to the HIV-1 Env protein. Finally, the membrane was reacted with an IRDye800 conjugated affinity purified goat anti-human IgG antibody (Rockland Immunochemicals; Gilbertsville, Pa.) and an Alexa-Fluor 680 conjugated goat anti-mouse IgG antibody (Invitrogen; Carlesbad, Calif.). Fluorescence was detected and the density of Env protein and p24 bands was determined with an Odyssey Infrared Imaging system (LiCor Biosciences; Lincoln, Nebr.). The level of Env protein in the transfected cells was expressed as fold differences over the amount of p24 Gag antigen in the same cells.

Sequence Analysis.

Sequences of SGA env amplicons were obtained by cycle-sequencing and dye terminator methods with an ABI 3730×1 genetic analyzer (Applied Biosystems; Foster City, Calif.). Individual sequence fragments for each env SGA were assembled and edited using the Sequencher program 4.7 (Gene Codes; Ann Arbor, Mich.). The env sequences were aligned using CLUSTAL W [41], and Genecutter (www.hiv.lanl.gov) for obtaining codon aligned files that could be used for signature analysis. The phylogenetic trees were constructed with either Neighbor joining or Maximum likelihood methods, as specified in the legends.

Hierarchical Clustering Analysis.

The neutralizing potential of plasma (the log reciprocal dilution ID₅₀ scores) was organized into patterns of similar neutralizing potency when tested against the panel of envelopes used in this study using the Los Alamos National Lab web-based heatmap interface (http://www.hiv.lanl.gov/content/sequence/HEATMAP/heatmap.html) and the statistical package R [42] The test panel of envelopes was resampled 10,000 times using a random-with-replacement bootstrap strategy, and this identified two robust and distinctive clusters recurring in 96% of the bootstrap samples: one group with little cross-neutralizing potential and another group with the ability to cross-neutralize multiple heterologous C clade viruses [43]. These two groups were used for subsequent signature analysis. The same strategy was used to identify statistically robust clusters of the Envs with like-neutralizing susceptibility. Clusters with high bootstrap values typically only included a few Envs each, and these Env clusters were typically found within a single subject. Therefore, we decided to organize Env pseudoviruses by individual in heatmap figure, while retaining the information regarding significant bootstrap clusters, shown superimposed onto the figure.

Signature Analysis.

We first divided the envelope sequences into the two groups, based on heatmap clustering patterns, that indicated whether they were derived from an individual with plasma that had a cross-reactive neutralizing profile or a weakly neutralizing profile. Alignments used for signature analysis were generated with GeneCutter (www.hiv.lanl.gov) to provide codon-aligned DNA for phylogenetic analysis. Phylogenetically corrected methods were used to identify all signature sites. Phylogenetic correction is critical because observed patterns in data can result either from correlations imposed by the initial historical emergence of a lineage of viruses (founder effects), or in the case of HIV-1, be a consequence of recent biological interactions. Not accounting for founder effects can lead to erroneous statistical conclusions [44]. The sequence of the virus depends on its full evolutionary history, while causal correlations are manifest in correlations with recent changes. The separation of the two effects, i.e. a phylogenetic correction, enables one to estimate the impact recent changes on phenotype. This requires statistical reconstruction the genealogical relationships between the viruses and an estimate of ancestral states of the viruses. We implemented this through maximum likelihood phylogenies, and tested for phenotypic associations with mutational patterns based on change or stasis in a given amino acid, when compared to the most recent common ancestor, adapting the phylogenetically corrected Fisher's exact methods first developed in Bhattacharya et al [44]. We screened the mutational pattern of each amino acid found every column in the alignment. To correct for multiple tests we used a q-value to assess the false discovery rate [45]. The four strongest associations found each had had a p-value of 0.03, and q-value of 0.27, thus is it is like that at least one of the signatures identified is a false positive, and only borderline significance was obtained overall, hence these signatures should be considered in a hypothesis-raising framework, as potentially interesting sites that merit further investigation.

Nucleotide Sequences Accession Numbers.

GenBank accession numbers off all env gene sequences are GU329048-GU329523.

### Results

Genetic Analysis of Full-Length env Genes.

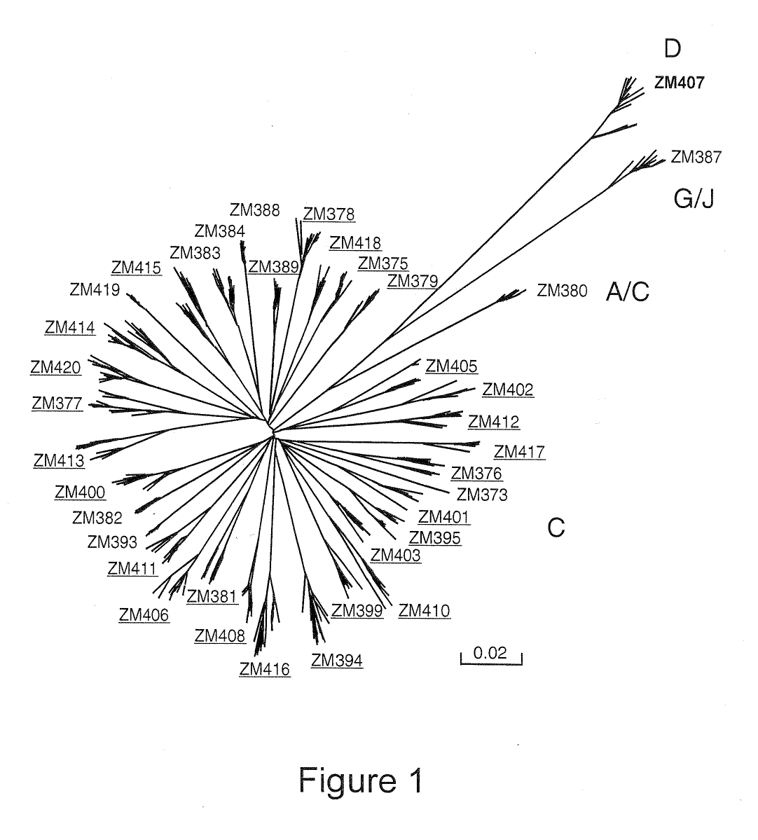

Plasma samples were collected from 50 CHI individuals in Ndola, Zambia in 2005. A total of 474 complete env genes were obtained from 37 of 50 subjects by SGA. Negative PCR results for the remaining 13 subjects were due to either low or undetectable plasma viral loads. An average of thirteen env SGAs (range 5-23) were sequenced from each of the 37 PCR-positive subjects (Table 1). Phylogenetic analysis showed that a majority of subjects (n=34) were infected with HIV-1 subtype C (FIG. 1). Three others were infected with either subtype D, an A/C recombinant (subtype C in the most central region) or a G/J recombinant. Monophyletic trees were observed in 11 individuals (type I), whereas two distinct env lineages (type II) were found in 26 individuals (70%) (FIG. 1). This frequency of two discrete lineages is much higher than the 20%-30% frequency observed in acute HIV-1 subtype B and C infections described previously [37,46] and in subtype B CHI individuals (unpublished data).

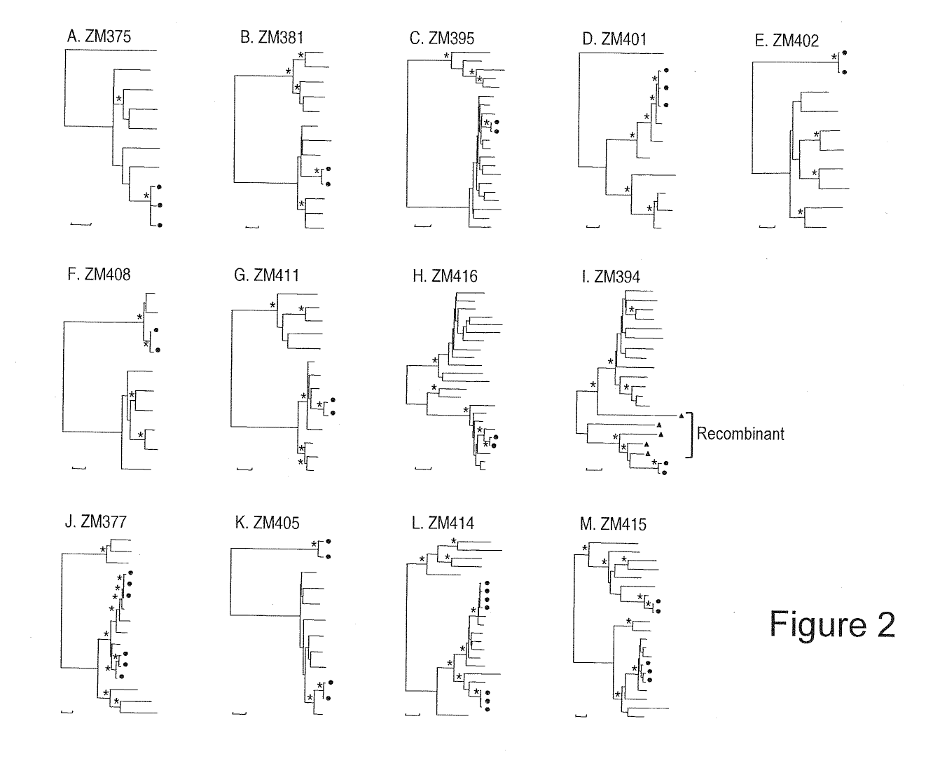

Identical or closely related env sequences were identified in 13 of 37 (35%) of subjects (Table 2), suggesting clonal expansion of minority viral populations in the HIV-1 infected individuals. In these subjects, clonally expanded Envs comprised 9-38% of the sampled env population. Sequences in each population of clonally expanded envelopes formed a tight cluster with zero or limited branch lengths in the phylogenetic trees (FIG. 2); this tight clustering was supported by high bootstrap values (>85%). Nine subjects had one clonally expanded env variant (FIG. 2A-I), whereas an additional four subjects (ZM377, ZM405, ZM414, and ZM415) had two independent clonally expanded populations (FIG. 2J-M). Clonally expanded variants in two subjects (ZM402 and ZM405) formed new lineages (FIGS. 2E and 2J). Sequences closely related to, but beginning to diverge from the clonally expanded variant were observed in four subjects (ZM377, ZM401, ZM414, and ZM415), (FIGS. 2D, J, L and M). Recombinant env sequences were detected between clonally expanded viruses and other viruses in ZM394 (FIG. 2I). These latter recombinants contributed to a substantial increase in diversity of the viral population in this subject. When recombinant sequences were excluded from the phylogenetic tree, the clonally expanded viruses represented an emergence of a new viral lineage in the subject (FIG. 2I).

Functional Analysis of the env Quasispecies in Infected Individuals.

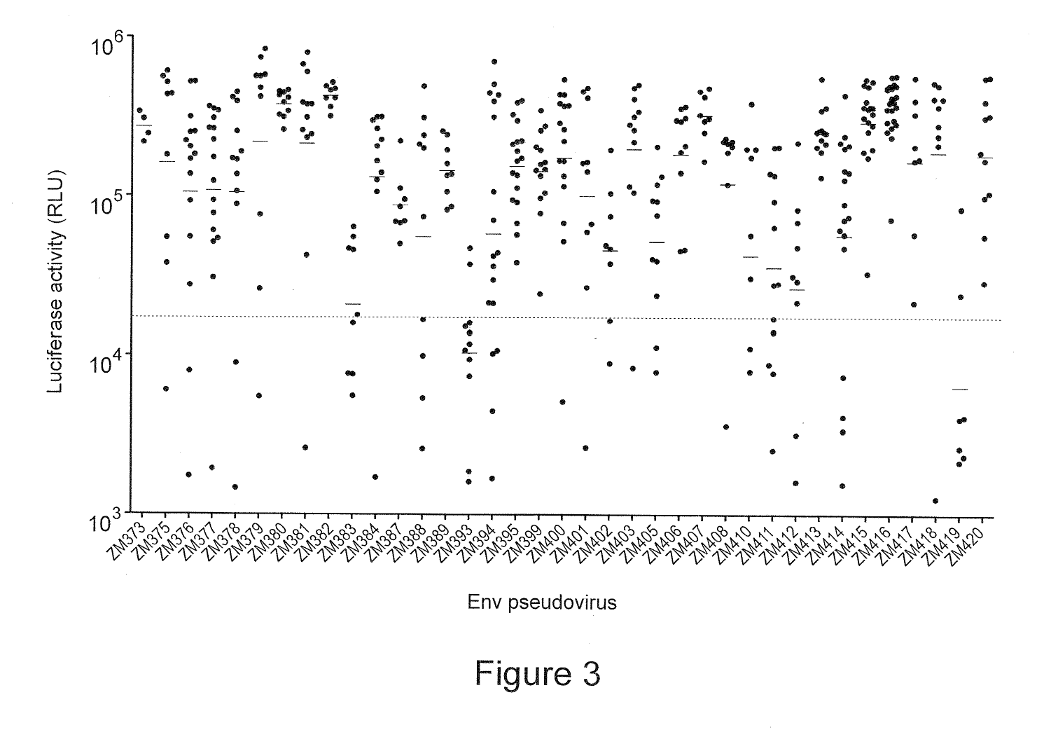

A pPCR method that adds a CMV promoter to SGA amplicons [38] was used for high throughput functional screening of multiple env genes from each infected individual. Env-pseudotyped viruses were generated by cotransfecting 293T cells with pPCR products and an Env-defective backbone plasmid (SG3Δenv). Infectivity was measured by luciferase activity in TZM-bl cells. Out of a total of 474 Envs examined, 377 (80%) were found to be functional (Table 1). All newly characterized env genes were functional in eight subjects, whereas in a few subjects only a small fraction of any genes were functional (10% and 18% for ZM419 and ZM393, respectively). In three individuals (ZM373, ZM380 and ZM382), all Env-pseudotyped viruses were highly infectious and differed in infectivity by only a few folds (FIG. 3). These latter env sequences comprised a monophyletic virus group in each subject. Notably, infectivity of Env-pseudotyped viruses from most other individuals was highly variable (1-2 log differences in luciferase activity).

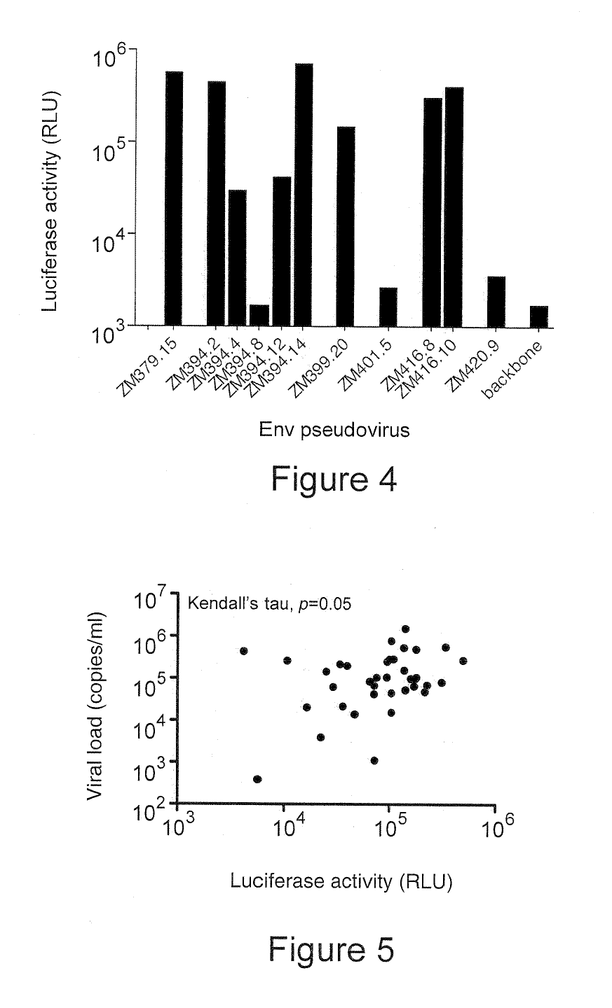

Recombinant genes were found in 11 env sequences from 6 individuals. Examination of their functionality showed that 8 (73%) were biologically functional while the other three were not (FIG. 4). ZM420.9 was not functional due to a reading frame shift caused by one nucleotide insertion. The result suggested that while some recombinants might gain biological advantages, others might be lethal and result in nonfunctional genes. For example, in ZM394, the recombinant viruses between the clonal expansion viruses and other viruses resulted in different levels of infectivity, ranging from nonfunctional to highly infectious (FIGS. 2I and 4). However, recombinant sequences were not particularly enriched for inactive viruses relative to non-recombinant (Fisher's exact test, p=0.4). Twenty-six individuals were infected with two lineage viruses, which are separated from each other in the phylogenetic tree analysis (FIG. 1). However, no significant differences in infectivity of Env-pseudoviruses were observed between different lineage viruses in the same patient (data not shown).

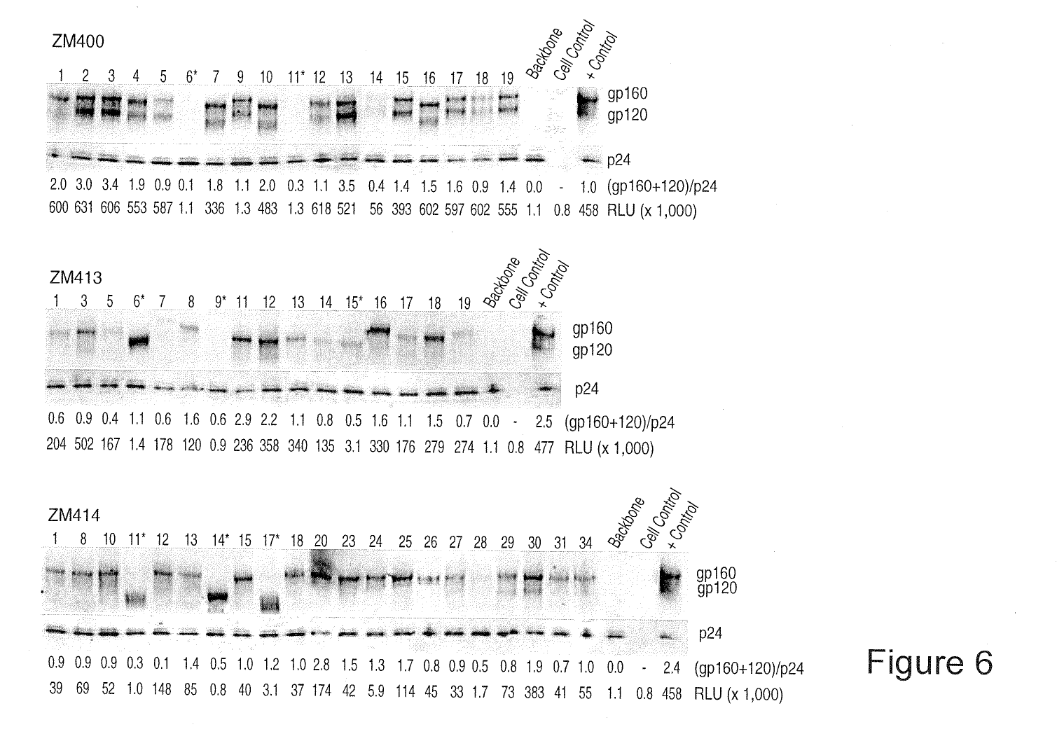

Given the range of variability in Env function, it was of interest to determine whether the level of Env infectivity in pseudovirus assays was associated with either protein expression levels or with plasma viral RNA load. As shown in FIG. 5, a trend toward a positive correlation was seen between pseudovirus infectivity and viral load (p=0.05). Protein expression in transfected 293T cells was quantified by Western blot for multiple env SGAs from three subjects (ZM400, ZM413 and ZM414) whose Env clones demonstrated a substantial range in infectivity. To control for variation between independent transfections, Env expression was standardized to p24 Gag protein expression in the same cells. The sizes and processing cleavage efficiency of gp160 into gp120 were different among tested env genes. While all Envs showed little size variation in ZM414 (except the prematurely truncated ones), others varied in both ZM400 and ZM413 (FIG. 6). The ectodomain gp120 was observed for all expressed Envs in ZM400, but most were uncleaved gp160 in ZM413 and ZM414. Infectivity of Env-pseudoviruses was found not to correlate to the level of Env expression in each individual.

These results suggested that variation in infectivity was not determined by expression levels, protein sizes or the cleavage efficiency of gp160 in transfected cells. It is possible that the amount of Envs that were sufficient for infection in transfected cells was above the threshold required for the production of fully infectious pseudoviruses since only a small number of Env spikes are present on mature HIV-1 particles [47,48]. Non-infectious pseudoviruses were associated with either the absence of Env expression or with the expression of truncated Envs; these cases were explained by either premature stop codons or frame-shifting deletions by sequence analysis.

Autologous and Heterologus Neutralization.

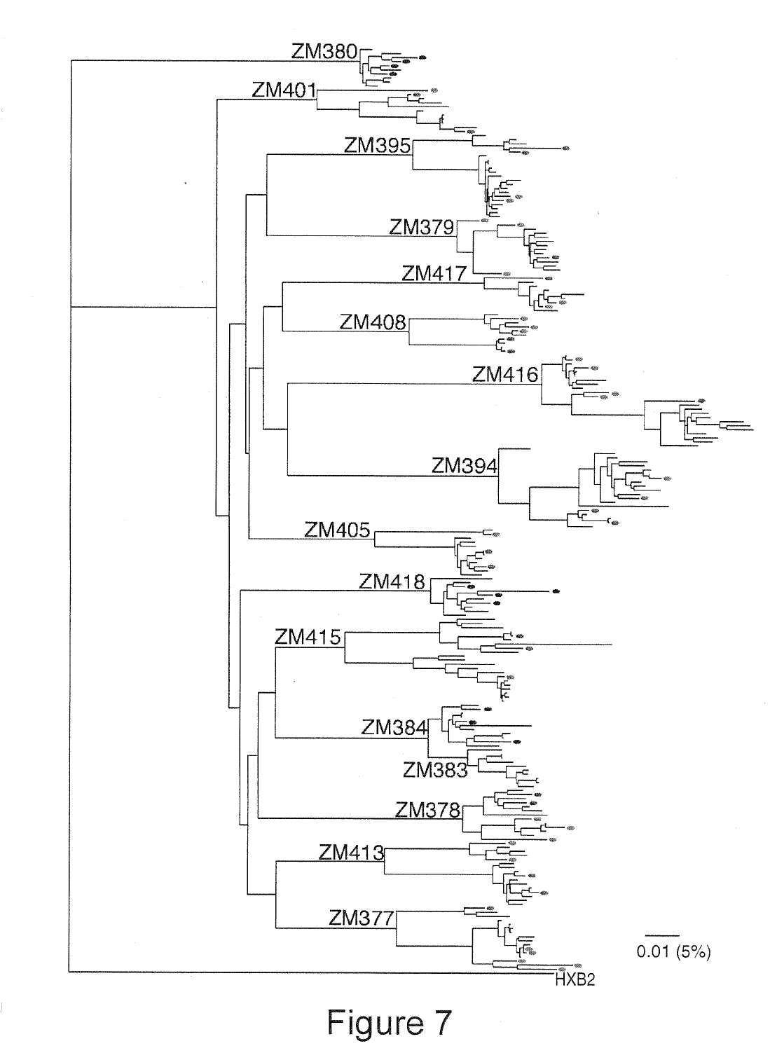

A checkerboard-style dataset of the neutralizing activity in autologous and heterologous plasma samples was generated by assaying plasma samples against multiple representative Env-pseudotyped viruses from each subject. Because of the limited supply of plasma, it was only possible to perform neutralization assays with 60 Env-pseudotyped viruses from 15 subjects (14 subtype C and one A/C recombinant) using plasma samples from these 15 subjects plus one additional plasma sample (ZM383). Multiple env genes (range 3-6) from each subject were selected to represent the viral population as seen in a Maximum-likelihood tree analysis (FIG. 7). Three or four env genes were chosen for monophyletic viruses, whereas 1-3 env genes were selected from each lineage in individuals with multiple Env lineages.

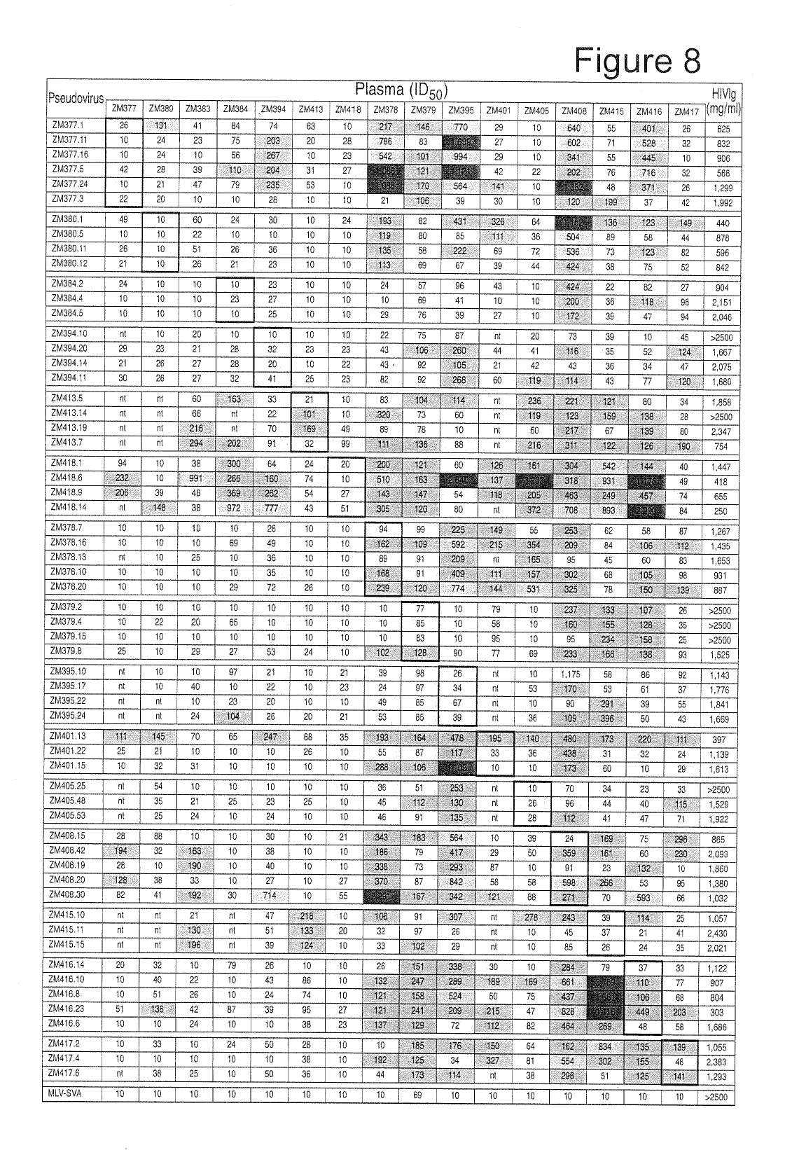

Neutralization results are summarized in FIG. 8. A majority of virus/plasma combinations (75%) were positive with neutralization titers of 20-3,769, where approximately one-third of the combinations resulted in a neutralization titer >100 (FIG. 8). Variable levels of autologous virus neutralization by contemporaneous plasma (i.e., plasma obtained at that same time point as Env) was detected in all but one subject (ZM380). The neutralization potency was relatively weak in most cases; however, titers >100 were detected against at least one variant in the autologous virus quasispecies of 7 subjects (ZM378, ZM379, ZM401, ZM408, ZM413, ZM416 and ZM417). In several cases, substantial differences were seen between multiple env genes from the same subject. For example, titers of autologous neutralization against multiple env variants in subject ZM408 ranged from 24 to 598, and in subject ZM416 they ranged from 37 to 449.

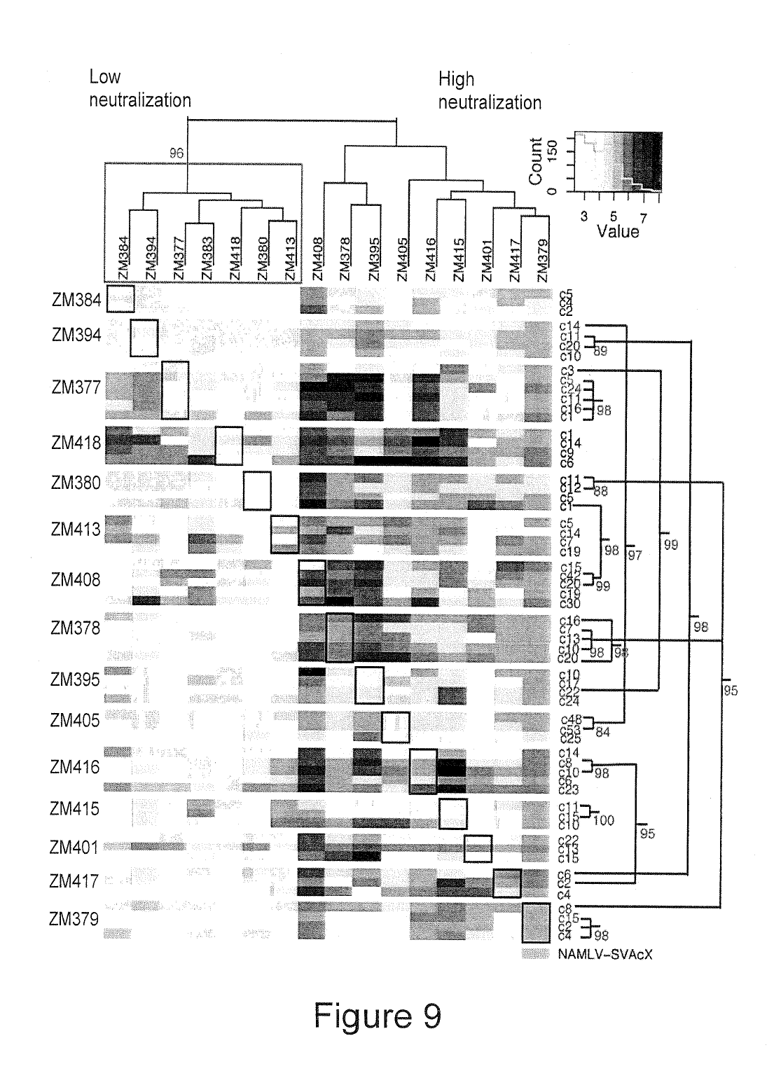

All Env-pseudotyped viruses were then assayed for neutralization susceptibility by heterologous plasma. Plasma samples were segregated into low (L) and high (H) neutralization potency groups by heatmap analysis (FIG. 9). Statistically supported clustering of plasma samples was observed, with the distinctive low-activity cluster boxed on the left. Overall, seven plasma samples had limited cross-neutralizing activity. However, the other nine plasma samples possessed moderate to potent cross-reactive neutralization activity (FIG. 8 and FIG. 9). Some plasmas (ZM408, ZM378, ZM395 and ZM379) neutralized nearly all 60 pseudoviruses from the 15 infected individuals.

Neutralization Signature Analysis.

The identification of plasma samples with potent and cross-reactive Nabs prompted us to ask if there were signature sequences present among the Envs from those subjects that associated with the potent neutralizing activity. Because each plasma sample represented one value per individual, we used only the consensus sequence of all variants within an individual for the signature study, thereby capturing the most common amino in each position found in each subject. We scanned each position in the alignment using the Maximum likelihood tree correction method previously described [44] to look for amino acids associated with either strong or weak subtype C cross-reactive neutralization responses, based on the two distinctive clusters shown in the heatmap (FIG. 9). Because of multiple test issues (a test was done for every site in Env), we used a q-value to estimate the false discovery rate, based on the distribution of p-values [45].

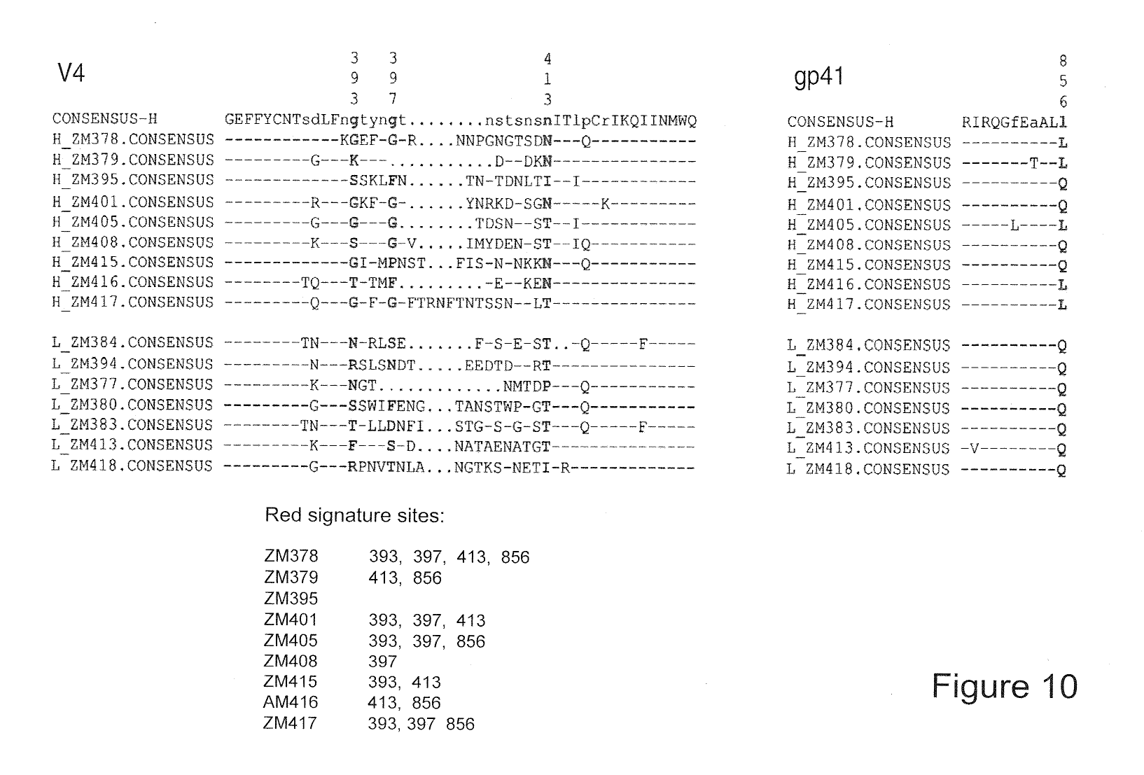

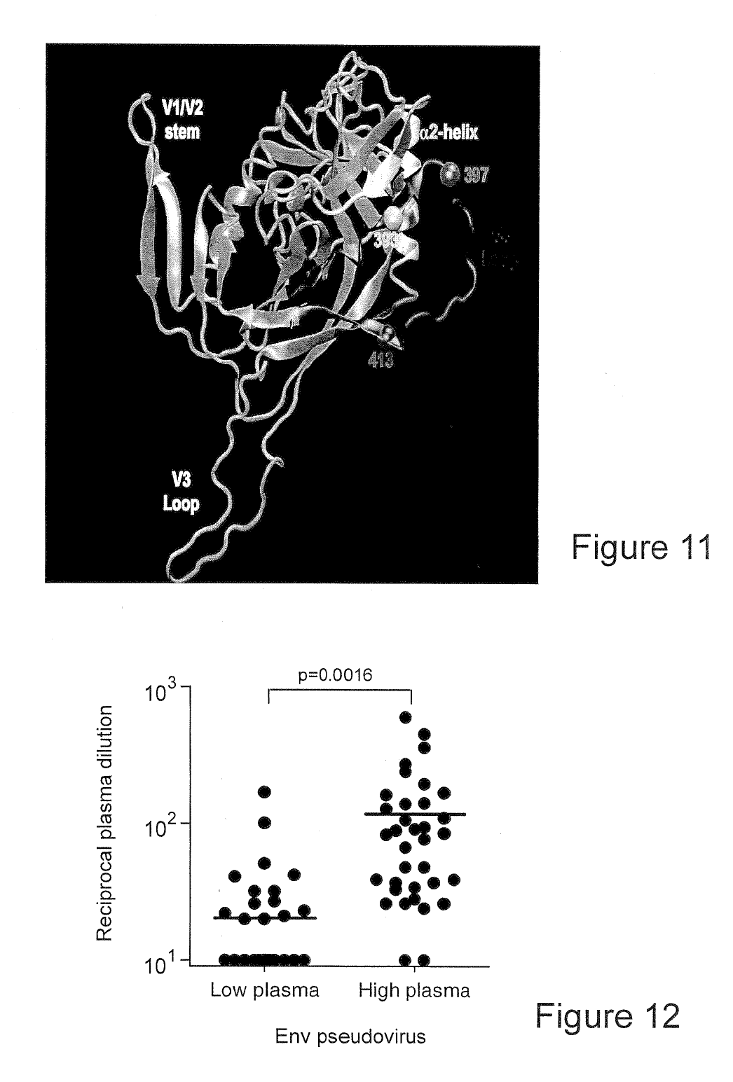

Four signature sites were identified: 3 in gp120 and one in gp41. Each site had a p-value of 0.03, but a q-value of 0.27; thus it may be that one or more of the signatures identified were false positives (FIG. 10). We include them here in a hypothesis-raising mode because they displayed the strongest trends towards significance. Intriguingly, three of the four signature sites were found in the V4 region (393G, 397G, and 413N). The V4 region, in association with the C3 alpha-2 helical domain, is thought to contribute to patterns of neutralization susceptibility in subtype C viruses [18,49,50] (FIG. 11). The fourth signature was found at the very end of the gp41 cytoplasm domain and consisted of a lysine (L) in position 856. The most intriguing signature was position 413N, which is part of an N-linked glycosylation site signature that was independently identified to be associated with good serological Nab breadth in a multi-subtype study of envelopes isolated from individuals who had elicited particularly potent neutralizing antibodies versus those that had weak responses (manuscript in preparation, SG, BH, FG, DM and BK). Thus Envs that stimulated more cross-reactive Nabs tended to have mutated towards an Asn (N) in position 413. They also tended to have mutations towards Gly (G) in positions 393 and 397, although 397 was in a highly variable region that is difficult to align with confidence.

Interestingly, autologous neutralizing activity was significantly higher (p=0.0016) in the H plasma group than in the L plasma group of heterologous neutralization potencies (FIG. 12). In other words, plasmas that possessed stronger neutralizing activity against heterologous viruses more potently neutralized the contemporaneous autologous virus. In addition, positions 393, 397, and 413 were found to be highly variable within single individuals, indicative of recurrent immune pressure in different infection on theses site. Collectively, these data suggest that signature patterns in the V4 loop may be important for the stimulation of potent and cross-reactive neutralizing antibodies against subtype C viruses.

In summary, we characterized multiple HIV-1 env genes in each of 37 individuals using SGA and pPCR methods, Sequence and functional analysis of the env gene quasispecies in each individual showed multiple lineages of viruses, frequent clonal expansions, a trend towards a positive correlation between viral loads and the infectivity of Env pseudoviruses, highly variable infectivity among env gene quasispecies populations, and signature amino acids in the V4 region of sequences in plasma with high levels of neutralization activity. Identification of such neutralization signatures may have implications for development of effective HIV-1 vaccines.

As expected for chronic HIV-1 infection, a highly divergent viral quasispecies population was detected in the subject studies here. Intra-subject env gene diversity was as high as 8% in this study. Using our recently established pPCR method, we were able to characterize the phenotypic properties of a large number of env genes from each HIV-1 infected individual by generating pseudoviruses without cloning SGA PCR products. Analysis of the infectivity of 474 Env pseudoviruses from 37 individuals revealed that a high percentage (80%) of the env genes were functional. This is consistent to our previous report with a smaller number of env genes [38] but much higher than the reported rate (10%) from env gene populations characterized by bulk PCR from PBMC [25]. The different rates were likely due to differences in methods and sample sources used to obtain env genes. Functional analysis showed that the infectivity of Env pseudoviruses from the same individuals varied significantly (1-2 logs) in most individuals. This is similar to plasma derived env genes from one individual [51], but different from PBMC derived env genes which showed uniform infectivity from the same individual [25]. Examination of Env proteins in the transfected cells showed that the variation of infectivity among Env pseudoviruses in each individual was not correlated with in vitro expression levels. With an intact env open reading frame, the non-functionality of the env genes may be caused by mutations that affect either incorporation into virus particles or poor interaction with CD4 and coreceptors on target cells. Interestingly, a positive correlation trend was observed between the levels of infectivity of Env-pseudoviruses and viral loads in the donor subjects, suggesting that viruses with more infectious env genes may lead to a high level of viral loads in vivo.

Sequence analysis showed that 13 of 37 individuals (35%) carried one or two clusters of closely related sequences among the quasispecies viral populations. These sequences, unique to each individual and conserved within that individual, were identical or nearly identical to each other, Since each sequence was obtained from independent PCR by SGA, they represent in vivo derived independent viral genomes. The env sequences related to but beginning to diverge from the identical or nearly identical sequences, as well as recombinants between these conserved sets and more diverse viruses from that individual, indicate that there is clonal expansion of some viruses in the infected individuals. Overall, they accounted for 9-38% of viral population. Clonal expansion of some virus species was found in different tissues in HIV-1 infected individuals [52]. It is not clear what the sources are for clonally expanded viruses in plasma. It is possible that clonally expanded viruses have an advantage due to replication fitness or immune escape mutations. Since clonal expansion viruses contain amino acids that differ from those in viruses in the major viral population, and because they can recombine with other viruses (FIG. 2I), they might play a role in evasion of immune responses elicited by viruses of the majority population. A longitudinal study of multiple time point samples will be required to test this hypothesis.

An analysis of checkerboard neutralization data with multiple representative env genes from each individual using autologous and heterologous plasma samples revealed a signature of three amino acids that associated with potent neutralizing plasmas. Glycines associated with signature sites 393 and 397 in the V4 loop of gp120 could impact antibody neutralization in several ways. Firstly, the loss of electrostatic charge associated with side chains can perturb antibody binding by modulating the electrostatic potential of the cognate epitope. Secondly, due to their lack of side chains, glycines exhibit higher flexibility that might affect conformational motions in the V4 loop. Flexibility and less charge repulsion due to glycines at positions 393 and 397 might allow V4 to sample a wide variety of conformations. In contrast, the signature 413N might play a role by mediating N-linked glycosylation and potentially inhibiting V4 loop movement by steric hindrence.

Site 413 is proximal to the 17b and CCRS binding sites, and is adjacent to the proximal binding motif RIKQ (HXB2 residues 419-422). A recent glycopeptide mass spectroscopy (MS) study found that glycans were indeed attached at this site in the group M consensus HIV-1 envelope, CON-S gp140 [53]. Both high-resolution methods, online high performance liquid chromatography-electrospray ionization mass spectrometry (HPLC/ESI-MS) and offline HPLC followed by matrix-assisted laser desorption/ionization mass spectrometry (MALDI-MS) determined that the potential glycosylation site 413 is in fact glycosylated. It is, however, not clear how a glycosylation at this position is associated with induction of higher levels of neutralizing antibody titers in vivo. This position is quite variable; indeed the glycosylation site could be enriched in Envs associated with potent neutralization activity because it is selected against by a strong response rather than being required to elicit a strong response.

In a recent study, a neutralization signature was investigated in subtype C sera using 36 sera against a panel of 5 subtype B and 5 C subtype viruses [20]. No clear clustering of serological patterns was found to enable tracking of the signature patterns. However, a correlation between the V1-V4 loop length and the cross-subtype neutralizing potential was observed. In the current study, we did not find evidence for such a correlation when comparing the total V1-V4 lengths or the lengths of V1, V2, and V4 separately by comparing the of lengths of the consensus Env from each individual grouped by the high and low neutralization plasma using a Wilcoxon rank statistic. It could have simply because that the number of the samples was underpowered to show this effect. The other possibility is that all samples in the current study were from CHI individuals in whom the length of variable loops had already become longer during the evolution of immune evasion.

Previous studies also showed that the alpha-2 helix was under strong selection pressure [50] and this region was found associated with resistance to autologous neutralization of subtype C viruses [49], The V4 region is immediately downstream from the alpha-2 helix and they are structurally close to each other. It was also found that the C3V4 region is likely responsible for inducing autologous and heterologous Nabs [18]. The newly identified three signature amino acids were located in the vicinity of the previously reported region targeted by Nabs, suggesting that tertiary structure may play an important role in the induction of Nabs against subtype C viruses. Identification of this potential signature may help to design a new generation Env immunogens that elicit potent and broadly reactive Nabs, at least for subtype C which is the most prevalent HIV-1 subtype in the world.

### REFERENCES

1. Burton D R, Desrosiers R C, Doms R W, Koff W C, Kwong P D, et al, (2004) HIV vaccine design and the neutralizing antibody problem. Nat Immunol 5: 233-236.
2. Haynes B F, Montefiori D C (2006) Aiming to induce broadly reactive neutralizing antibody responses with HIV-1 vaccine candidates. Expert Rev Vaccines 5: 347-363.
3. Korber B T, Letvin N L, Haynes B F (2009) T-cell vaccine strategies for human immunodeficiency virus, the virus with a thousand faces. J Virol 83: 8300-8314.
4. Hemelaar J, Gouws E, Ghys P D, Osmanov S (2006) Global and regional distribution of HIV-1 genetic subtypes and recombinants in 2004. Aids 20: W13-23.
5. Desrosiers R C (2004) Prospects for an AIDS vaccine. Nat Med 10: 221-223.
6. Trkola A, Pomales A B, Yuan H, Korber B, Maddon P J, et al. (1995) Cross-clade neutralization of primary isolates of human immunodeficiency virus type 1 by human monoclonal antibodies and tetrameric CD4-IgG. J Virol 69: 6609-6617.
7. Zwick M B, Labrijn A F, Wang M, Spenlehauer C, Saphire E O, et al. (2001) Broadly neutralizing antibodies targeted to the membrane-proximal external region of human immunodeficiency virus type 1 glycoprotein gp41. J Virol 75: 10892-10905.
8. Moore J P, McCutchan F E, Poon S W, Mascola J, Liu J, et al. (1994) Exploration of antigenic variation in gp120 from clades A through F of human immunodeficiency virus type 1 by using monoclonal antibodies. J Virol 68: 8350-8364.
9. Binley J M, Wrin T, Korber B, Zwick M B, Wang M, et al. (2004) Comprehensive cross-clade neutralization analysis of a panel of anti-human immunodeficiency virus type 1 monoclonal antibodies. J Virol 78: 13232-13252.
10. Lin G, Nara P L (2007) Designing immunogens to elicit broadly neutralizing antibodies to the HIV-1 envelope glycoprotein, Curr HIV Res 5: 514-541.
11. Stamatatos L, Morris L, Burton D R, Mascola J R (2009) Neutralizing antibodies generated during natural HIV-1 infection: good news for an HIV-1 vaccine? Nat Med.
12. Sather D N, Armann J, Ching L K, Mavrantoni A, Sellhom G, et al. (2009) Factors associated with the development of cross-reactive neutralizing antibodies during human immunodeficiency virus type 1 infection. J Virol 83: 757-769.
13. Binley J M, Lybarger E A, Crooks E T, Seaman M S, Gray E, et al. (2008) Profiling the specificity of neutralizing antibodies in a large panel of plasmas from patients chronically infected with human immunodeficiency virus type I subtypes B and C. J Virol 82: 11651-11668.
14. Dhillon A K, Dormers H, Pantophlet R, Johnson W E, Decker J M, et al. (2007) Dissecting the neutralizing antibody specificities of broadly neutralizing sera from human immunodeficiency virus type 1-infected donors. J Virol 81: 6548-6562.
15. Li Y, Migueles S A, Welcher B, Svehla K, Phogat A, et al. (2007) Broad HIV-1 neutralization mediated by CD4-binding site antibodies. Nat Med 13: 1032-1034.
16. Shen X, Parks R J, Montefiori D C, Kirchherr J L, Keele B F, et al. (2009) In vivo gp41 antibodies targeting the 2F5 monoclonal antibody epitope mediate human immunodeficiency virus type 1 neutralization breadth. J Virol 83: 3617-3625.
17, Rong R, Li B, Lynch R M, Haaland R E, Murphy M K, et al. (2009) Escape from autologous neutralizing antibodies in acute/early subtype C HIV-1 infection requires multiple pathways. PLoS Pathog 5: e1000594.
18. Moore P L, Gray E S, Choge I A, Ranchobe N, Mlisana K, et al. (2008) The c3-v4 region is a major target of autologous neutralizing antibodies in human immunodeficiency virus type 1 subtype C infection. J Virol 82: 1860-1869.
19. Moore P L, Ranchobe N, Lambson B E, Gray E S, Cave E, et al. (2009) Limited neutralizing antibody specificities drive neutralization escape in early HIV-1 subtype C infection. PLoS Pathog 5: e1000598.
20. Rademeyer C, Moore P L, Taylor N, Martin D P, Choge I A, et al. (2007) Genetic characteristics of HIV-1 subtype C envelopes inducing cross-neutralizing antibodies. Virology 368: 172-181.
21. Kulkami S S, Lapedes A, Tang H, Gnanakaran S, Daniels M G, et al. (2009) Highly complex neutralization determinants on a monophyletic lineage of newly transmitted subtype C HIV-1 Env clones from India. Virology 385: 505-520.
22. Walker L M, Phogat S K, Chan-Hui P Y, Wagner D, Phung P, et al, (2009) Broad and Potent Neutralizing Antibodies from an African Donor Reveal a New HIV-1 Vaccine Target. Science.
23. Derdeyn C A, Decker J M, Bibollet-Ruche F, Mokili J L, Muldoon M, et al. (2004) Envelope-constrained neutralization-sensitive HIV-1 after heterosexual transmission. Science 303: 2019-2022.
24. Wei X, Decker J M, Wang S, Hui H, Kappes J C, et al. (2003) Antibody neutralization and escape by HIV-1. Nature 422: 307-312,
25. Cham F, Zhang P F, Heyndrickx L, Bouma P, Zhong P, et al. (2006) Neutralization and infectivity characteristics of envelope glycoproteins from human immunodeficiency virus type 1 infected donors whose sera exhibit broadly cross-reactive neutralizing activity. Virology 347: 36-51.
26. Zhang P F, Cham F, Dong M, Choudhary A, Bouma P, et al. (2007) Extensively cross-reactive anti-HIV-1 neutralizing antibodies induced by gp140 immunization. Proc Nati Acad Sci USA 104: 10193-10198.
27. Cordonnier A, Montagnier L, Emerman M (1989) Single amino-acid changes in HIV envelope affect viral tropism and receptor binding. Nature 340: 571-574.
28. Kalia V, Sarkar S, Gupta P, Montelaro R C (2005) Antibody neutralization escape mediated by point mutations in the intracytoplasmic tail of human immunodeficiency virus type 1 gp41. J Virol 79: 2097-2107.
29. LaBranche C C, Sauter M M, Haggarty B S, Vance P J, Romano J, et al. (1995) A single amino acid change in the cytoplasmic domain of the simian immunodeficiency virus transmembrane molecule increases envelope glycoprotein expression on infected cells. J Virol 69: 5217-5227.
30. Morris J F, Sternberg E J, Gutshall L, Petteway S R, Jr., Ivanoff L A (1994) Effect of a single amino acid substitution in the V3 domain of the human immunodeficiency virus type 1: generation of revertant viruses to overcome defects in infectivity in specific cell types. J Virol 68: 8380-8385.
31. Shimizu N, Haraguchi Y, Takeuchi Y, Soda Y, Kanbe K, et al. (1999) Changes in and discrepancies between cell tropisms and coreceptor uses of human immunodeficiency virus type 1 induced by single point mutations at the V3 tip of the env protein. Virology 259: 324-333.
32. Shioda T, Oka S, Ida S, Nokihara K, Toriyoshi H, et al, (1994) A naturally occurring single basic amino acid substitution in the V3 region of the human immunodeficiency virus type 1 env protein alters the cellular host range and antigenic structure of the virus. J Virol 68: 7689-7696.
33, Palmer S, Kearney M, Maldarelli F, Halvas E K, Bixby C J, et al. (2005) Multiple, linked human immunodeficiency virus type 1 drug resistance mutations in treatment-experienced patients are missed by standard genotype analysis. J Clin Microbiol 43: 406-413.
34. Fang G, Zhu G, Burger H, Keithly J S, Weiser B (1998) Minimizing DNA recombination during long RT-PCR. J Virol Methods 76: 139-148.
35. Liu S L, Rodrigo A G, Shankarappa R, Learn G H, Hsu L, et al. (1996) HIV quasispecies and resampling. Science 273: 415-416.
36. Salazar-Gonzalez J F, Bailes E, Pham K T, Salazar M G, Guffey M B, et al. (2008) Deciphering human immunodeficiency virus type 1 transmission and early envelope diversification by single-genome amplification and sequencing. J Virol 82: 3952-3970.
37. Keele B F, Giorgi E E, Salazar-Gonzalez J F, Decker J M, Pham K T, et al. (2008) Identification and characterization of transmitted and early founder virus envelopes in primary HIV-1 infection. Proc Nati Acad Sci USA 105: 7552-7557.
38. Kirchherr J L, Lu X, Kasongo W, Chaiwe V, Mwananyanda L, et al. (2007) High throughput functional analysis of HIV-1 env genes without cloning. Virol Methods 143: 104-111.
39. Li M, Gao F, Mascola J R, Stamatatos L, Polonis V R, et al, (2005) Human immunodeficiency virus type 1 env clones from acute and early subtype B infections for standardized assessments of vaccine-elicited neutralizing antibodies, J Virol 79: 10108-10125.
40. Li M, Salazar-Gonzalez J F, Derdeyn C A, Morris L, Williamson C, et al. (2006) Genetic and neutralization properties of subtype C human immunodeficiency virus type 1 molecular env clones from acute and early heterosexually acquired infections in Southern Africa. J Virol 80: 11776-11790.
41. Thompson J D, Higgins D G, Gibson T J (1994) CLUSTAL W: improving the sensitivity of progressive multiple sequence alignment through sequence weighting, position-specific gap penalties and weight matrix choice. Nucleic Acids Res 22: 4673-4680.
42. R Development Core Team (2009) A Language and Environment for Statistical Computing. Vienna, Austria: R Foundation for Statistical Computing.
43. Shimodaira H (2004) Approximately unbiased tests of regions using multistep-multiscale bootstrap resampling. Annals of Statistics 32: 2616-2641.
44. Bhattacharya T, Daniels M, Heckerman D, Foley B, Frahm N, et al. (2007) Founder effects in the assessment of HIV polymorphisms and HLA allele associations. Science 315: 1583-1586.
45. Storey J D, Tibshirani R (2003) Statistical significance for genomewide studies. Proc Nati Acad Sci USA 100: 9440-9445.
46. Abrahams M R, Anderson J A, Giorgi E E, Seoighe C, Mlisana K, et al. (2009) Quantitating the multiplicity of infection with human immunodeficiency virus type 1 subtype C reveals a non-poisson distribution of transmitted variants. J Virol 83: 3556-3567.
47. Chertova E, Bess Jr J W, Jr., Crise B J, Sowder I R, Schaden T M, et al. (2002) Envelope glycoprotein incorporation, not shedding of surface envelope glycoprotein (gp120/SU), Is the primary determinant of SU content of purified human immunodeficiency virus type 1 and simian immunodeficiency virus. J Virol 76: 5315-5325.
48. Zhu P, Chertova E, Bess J, Jr., Lifson J D, Arthur L O, et al. (2003) Electron tomography analysis of envelope glycoprotein trimers on HIV and simian immunodeficiency virus virions. Proc Nati Acad Sci USA 100: 15812-15817.
49. Rong R, Gnanakaran S, Decker J M, Bibollet-Ruche F, Taylor J, et al. (2007) Unique mutational patterns in the envelope alpha 2 amphipathic helix and acquisition of length in gp120 hypervariable domains are associated with resistance to autologous neutralization of subtype C human immunodeficiency virus type 1. J Virol 81: 5658-5668.
50. Gnanakaran S, Lang D, Daniels M, Bhattacharya T, Derdeyn C A, et al. (2007) Clade-specific differences between human immunodeficiency virus type 1 clades B and C: diversity and correlations in C3-V4 regions of gp120. J Virol 81: 4886-4891,
51. Huang W, Toma J, Fransen S, Stawiski E, Reeves J D, et al. (2008) Coreceptor tropism can be influenced by amino acid substitutions in the gp41 transmembrane subunit of human immunodeficiency virus type 1 envelope protein. J Virol 82: 5584-5593.
52. Bull M E, Learn G H, McElhone S, Hitti J, Lockhart D, et al. (2009) Monotypic human immunodeficiency virus type 1 genotypes across the uterine cervix and in blood suggest proliferation of cells with provirus. J Virol 83: 6020-6028.
53. Irungu J, Go E P, Zhang Y, Dalpathado D S, Liao H X, et al. (2008) Comparison of HPLC/ESI-FTICR MS versus MALDI-TOF/TOF MS for glycopeptide analysis of a highly glycosylated HIV envelope glycoprotein. J Am Soc Mass Spectrom 19: 1209-1220.
54. Saitou N, Nei M (1987) The neighbor-joining method: a new method for reconstructing phylogenetic trees. Mol Biol Evol 4: 406-425.
55. Kimura M (1980) A simple method for estimating evolutionary rates of base substitutions through comparative studies of nucleotide sequences. J Mol Evol 16: 111-120.
56. Huang C C, Tang M, Zhang M Y, Majeed S, Montabana E, et al. (2005) Structure of a V3-containing HIV-1 gp120 core. Science 310: 1025-1028.

### TABLES

TABLE 1
Summary of SGAs and functional env genes from HIV-1 infected
individuals

| | | No. of | No. of functional | % of functional env |
|---|---|---|---|---|
| Patient ID | Subtype | SGAs | env genes | genes |
| ZM373 | C | 5 | 4 | 80 |
| ZM375 | C | 10 | 8 | 80 |
| ZM376 | C | 15 | 13 | 87 |
| ZM377 | C | 16 | 15 | 94 |
| ZM378 | C | 13 | 11 | 85 |
| ZM379 | C | 14 | 11 | 79 |
| ZM380 | A/C | 10 | 10 | 100 |
| ZM381 | C | 13 | 12 | 92 |
| ZM382 | C | 10 | 10 | 100 |
| ZM383 | C | 10 | 5 | 50 |
| ZM384 | C | 11 | 10 | 91 |
| ZM387 | G/J | 9 | 9 | 100 |
| ZM388 | C | 11 | 6 | 55 |
| ZM389 | C | 10 | 10 | 100 |
| ZM393 | C | 11 | 2 | 18 |
| ZM394 | C | 19 | 12 | 63 |
| ZM395 | C | 21 | 20 | 95 |
| ZM399 | C | 14 | 14 | 100 |
| ZM400 | C | 18 | 15 | 83 |
| ZM401 | C | 11 | 9 | 82 |
| ZM402 | C | 9 | 6 | 67 |
| ZM403 | C | 11 | 10 | 91 |
| ZM405 | C | 12 | 9 | 75 |
| ZM406 | C | 10 | 10 | 100 |
| ZM407 | D | 11 | 9 | 82 |
| ZM408 | C | 10 | 8 | 80 |
| ZM410 | C | 11 | 6 | 55 |
| ZM411 | C | 14 | 6 | 43 |
| ZM412 | C | 10 | 7 | 70 |
| ZM413 | C | 16 | 14 | 88 |
| ZM414 | C | 22 | 14 | 64 |
| ZM415 | C | 21 | 18 | 86 |
| ZM416 | C | 23 | 23 | 100 |
| ZM417 | C | 9 | 9 | 100 |
| ZM418 | C | 10 | 8 | 80 |
| ZM419 | C | 10 | 1 | 10 |
| ZM420 | C | 14 | 13 | 93 |
| Total | | 474 | 377 | 80 |

TABLE 2
Characterization of clonal expansion env sequences in HIV-1 infected
individuals

| | | Luciferase | % of | No. of amino acid |
|---|---|---|---|---|
| | | activity | total viral | differences among |
| Patient ID | SGA No. | (RLU) | population | env sequences |
| ZM375 | 1 | 434,052 | 30% | 3, 4, 5 |
| | 9 | 437,851 | | |
| | 11 | 610,112 | | |
| ZM377 (I) | 10 | 175,205 | 19% | 1, 2, 3 |
| | 13 | 310,710 | | |
| | 14 | 53,926 | | |
| ZM377 (II) | 11 | 354,745 | 19% | 1, 4, 5 |
| | 12 | 30,765 | | |
| | 16 | 77,427 | | |
| ZM378 | 7 | 802,968 | 15% | 3 |
| | 9 | 50,041 | | |
| ZM394 | 11 | 398,245 | 10% | 2 |
| | 18 | 21,273 | | |
| ZM395 | 14 | 327,566 | 10% | 1 |
| | 15 | 192,620 | | |
| ZM401 | 2 | 164,195 | 27% | 2 |
| | 19 | 66,826 | | |
| | 20 | 161,349 | | |
| ZM402 | 8 | 48,981 | 20% | 0 |
| | 12 | 8,985 | | |
| ZM405 (I) | 25 | 205,738 | 17% | 4 |
| | 44* | 1,393 | | |
| ZM405 (II) | 43 | 92,501 | 17% | 0 |
| | 53 | 118,441 | | |
| ZM408 | 15 | 118,979 | 20% | 3 |
| | 23* | 2,218 | | |
| ZM411 | 6 | 92,343 | 14% | 3 |
| | 9 | 203,082 | | |
| ZM413 | 5 | 229,782 | 13% | 3 |
| | 13 | 348,872 | | |
| ZM414 (I) | 1 | 47,219 | 18% | 0, 1 |
| | 10 | 70,754 | | |
| | 23 | 218,271 | | |
| | 28 | 4,134 | | |
| ZM414 (II) | 9 | 141,022 | 14% | 1 |
| | 20 | 240,243 | | |
| | 25 | 126,764 | | |
| ZM415 (I) | 1 | 510,266 | 16% | 4 |
| | 2 | 114,320 | | |
| | 26 | 553,315 | | |
| ZM415 (II) | 15 | 496,933 | 10% | 1 |
| | 27 | 297,937 | | |
| ZM416 | 3 | 391,763 | 9% | 2 |
| | 16 | 296,184 | | |
| Control | SG3Δenv | 1,127 | | |

Notes:
*Stop Codon;
I—phylogenetic cluster I;
II—phylogenetic cluster II

All documents and other information sources cited herein are hereby incorporated in their entirety by reference.

1. An HIV envelope protein comprising signature amino acids of ZM378, ZM379, ZM395, ZM401, ZM405, ZM408, ZM415, ZM416, and ZM417.

2. The protein according to claim 1 wherein said protein comprises signature aminos of ZM378.

3. The protein according to claim 1 wherein said protein is a gp160 or gp 140 protein.

4. The protein according to claim 1 wherein said protein comprises the amino acid sequence of ZM378 shown in FIG. 10.

5. An isolated nucleic acid encoding the protein according to claim 1.

6. The nucleic acid according to claim 5 wherein said nucleic acid is present in a vector.

7. The nucleic acid according to claim 6 wherein said vector is a viral vector.

8. A composition comprising the protein according to claim 1 and a carrier.

9. The composition according to claim 8 wherein said composition further comprises an adjuvant.

10. A method of inducing an immune response in a mammal comprising administering said protein according to claim 1 to said mammal in an amount sufficient to induce said response.

11. The method according to claim 10 wherein said mammal is a human.

12. An isolated antibody specific for said protein according to claim 1, or antigen binding fragment thereof.

13. A method of inhibiting infection of a mammalian cell by HIV-1 comprising contacting said cell with said antibody according to claim 12, or said fragment thereof, under conditions so that said inhibition is effected.

14. The method according to claim 13 wherein said cell is a human cell.

15. A composition comprising the nucleic acid according to claim 5 and a carrier.

16. The composition according to claim 15 wherein said composition further comprises an adjuvant.

17. A method of inducing an immune response in a mammal comprising administering said nucleic acid according to claim 5 to said mammal in an amount sufficient to induce said response.

18. The method according to claim 17 wherein said mammal is a human.


SEQUENCE LISTING (35 sequences)

SEQ ID NO: 1 - protein, 41 aa, Human immunodeficiency virus 1
GEFFYCNTSDLFNGTYNGTNSTSNSNITLPCRIKQIINMWQ

SEQ ID NO: 2 - protein, 45 aa, Human immunodeficiency virus 1
GEFFYCNTSDLFKGEFNGTRNNPGNGTSDNITLQCRIKQIINMWQ

SEQ ID NO: 3 - protein, 38 aa, Human immunodeficiency virus 1
GEFFYCNTSGLFNKTYNDTSDKNITLPCRIKQIINMWQ

SEQ ID NO: 4 - protein, 43 aa, Human immunodeficiency virus 1
GEFFYCNTSDLFNSSKLFNTNNTDNLTIITIPCRIKQIINMWQ

SEQ ID NO: 5 - protein, 43 aa, Human immunodeficiency virus 1
GEFFYCNTSRLFNGKFNGTYNRKDSSGNITLPCKIKQIINMWQ

SEQ ID NO: 6 - protein, 41 aa, Human immunodeficiency virus 1
GEFFYCNTSGLFNGTYNGTDSNSNSTITIPCRIKQIINMWQ

SEQ ID NO: 7 - protein, 44 aa, Human immunodeficiency virus 1
GEFFYCNTSKLFNSTYNGTVIMYDENNSTITIQCRIKQIINMWQ

SEQ ID NO: 8 - protein, 46 aa, Human immunodeficiency virus 1
GEFFYCNTSDLFNGIYMPNSTFISNNTNKKNITLQCRIKQIINMWQ

SEQ ID NO: 9 - protein, 40 aa, Human immunodeficiency virus 1
GEFFYCNTTQLFNTTTMFNETSKENITLPCRIKQIINMWQ

SEQ ID NO: 10 - protein, 49 aa, Human immunodeficiency virus 1
GEFFYCNTSQLFNGTFNGTFTRNFTNTSSNSNLTITLPCRIKQIINMWQ

SEQ ID NO: 11 - protein, 40 aa, Human immunodeficiency virus 1
GEFFYCNTTNLFNNTRLSEFNSTENSTLQCRIKQFINMWQ

SEQ ID NO: 12 - protein, 44 aa, Human immunodeficiency virus 1
GEFFYCNTSNLFNRSLSNDTEEDTDSNRTITLPCRIKQIINMWQ

SEQ ID NO: 13 - protein, 36 aa, Human immunodeficiency virus 1
GEFFYCNTSKLFNNGTNMTDPITLQCRIKQIINMWQ

SEQ ID NO: 14 - protein, 46 aa, Human immunodeficiency virus 1
GEFFYCNTSGLFNSSWIFENGTANSTWPNGTITLQCRIKQIINMWQ

SEQ ID NO: 15 - protein, 46 aa, Human immunodeficiency virus 1
GEFFYCNTTNLFNTTLLDNFISTGNSTGNSTITLQCRIKQFINMWQ

SEQ ID NO: 16 - protein, 45 aa, Human immunodeficiency virus 1
GEFFYCNTSKLFNFTYNSTDNATAENATGTITLPCRIKQIINMWQ

SEQ ID NO: 17 - protein, 46 aa, Human immunodeficiency virus 1
GEFFYCNTSGLFNRPNVTNLANGTKSTNETIIRLPCRIKQIINMWQ

SEQ ID NO: 18 - protein, 11 aa, Human immunodeficiency virus 1
RIRQGFEAALL

SEQ ID NO: 19 - protein, 11 aa, Human immunodeficiency virus 1
RIRQGFEAALL

SEQ ID NO: 20 - protein, 11 aa, Human immunodeficiency virus 1
RIRQGFETALL

SEQ ID NO: 21 - protein, 11 aa, Human immunodeficiency virus 1
RIRQGFEAALQ

SEQ ID NO: 22 - protein, 11 aa, Human immunodeficiency virus 1
RIRQGFEAALQ

SEQ ID NO: 23 - protein, 11 aa, Human immunodeficiency virus 1
RIRQGLEAALL

SEQ ID NO: 24 - protein, 11 aa, Human immunodeficiency virus 1
RIRQGFEAALQ

SEQ ID NO: 25 - protein, 11 aa, Human immunodeficiency virus 1
RIRQGFEAALQ

SEQ ID NO: 26 - protein, 11 aa, Human immunodeficiency virus 1
RIRQGFEAALL

SEQ ID NO: 27 - protein, 11 aa, Human immunodeficiency virus 1
RIRQGFEAALL

SEQ ID NO: 28 - protein, 11 aa, Human immunodeficiency virus 1
RIRQGFEAALQ

SEQ ID NO: 29 - protein, 11 aa, Human immunodeficiency virus 1
RIRQGFEAALQ

SEQ ID NO: 30 - protein, 11 aa, Human immunodeficiency virus 1
RIRQGFEAALQ

SEQ ID NO: 31 - protein, 11 aa, Human immunodeficiency virus 1
RIRQGFEAALQ

SEQ ID NO: 32 - protein, 11 aa, Human immunodeficiency virus 1
RIRQGFEAALQ

SEQ ID NO: 33 - protein, 11 aa, Human immunodeficiency virus 1
RVRQGFEAALQ

SEQ ID NO: 34 - protein, 11 aa, Human immunodeficiency virus 1
RIRQGFEAALQ

SEQ ID NO: 35 - protein, 4 aa, Artificial Sequence
  Description of Artificial Sequence Synthetic peptide
RIKQ